(19) United States
(12) Patent Application Publication
(10) Pub. No.: US 2012/0128749 A1
(43) Pub. Date: May 24, 2012

(54) COMPOSITION AND PROCESS FOR PRODUCTION THEREOF

(76) Inventors: Hideyasu Tsuji, Hiroshima (JP); Yasuhiro Tsuji, Hiroshima (JP); Toru Oka, Kofu-shi (JP); Shigeru Sugi, Minamikoma-gun (JP); Masumi Torii, Nakakoma-gun (JP); Haruka Miyao, Minamiarupusu-shi (JP); Yoshimitsu Nakayama, Minamiarupusu-shi (JP); Tomoyuki Torii, Nakakoma-gun (JP); Masahito Mori, Osaka (JP)

(21) Appl. No.: 13/361,150

(22) Filed: Jan. 30, 2012

Related U.S. Application Data
(63) Continuation of application No. PCT/JP2010/063316, filed on Aug. 5, 2010.

(30) Foreign Application Priority Data
Aug. 6, 2009 (JP) JP2009-183755

(51) Int. Cl. A01N 25/02; A01P 1/00; A01P 3/00; A01P 17/00; A01P 7/04; A61P 31/10; A61K 8/02; B08B 7/02; A61K 9/14; A61P 31/04; B82Y 40/00; B82Y 5/00; B82Y 99/00

(52) U.S. Cl. 424/405; 424/400; 510/438; 512/1; 134/1; 977/700; 977/906; 977/961; 977/840

(57) ABSTRACT

Disclosed are: a composition which enables the more effective development of the efficacy of a water-soluble drug in a solution containing the drug; and a dispersion in which a hydrophobic drug can be dispersed stably without requiring the use of any surfactant. Specifically disclosed are: a composition comprising ultra-fine bubbles having a mode particle size of 500 nm or less, a drug and water; and a process for producing a composition comprising ultra-fine bubbles having a mode particle size of 500 nm or less, a drug and water, which utilizes an ultra-fine bubble generation apparatus.

### RELATED APPLICATIONS

This application is a continuation of PCT/JP2010/063316 filed on Aug. 5, 2010, which claims priority to Japanese Application No. 2009-183755 filed on Aug. 6, 2009. The entire contents of these applications are incorporated herein by reference.

### FIELD OF THE INVENTION

The present invention relates to a composition comprising a large amount of ultrafine bubbles and a drug, a dispersion having a hydrophobic drug dispersed in water in absence of a surfactant, processes for producing the composition and the dispersion, as well as a detergent composition prepared from a specified recipe and a washing method that uses the detergent composition.

### BACKGROUND OF THE INVENTION

Recently, an apparatus has been developed that generates ultrafine bubbles commonly called nanobubbles. However, the use of this apparatus is limited to those applications where water that contains nanobubbles is used in washing operations or wastewater treatment and no studies have been made concerning drug-containing systems.

A method in which a chemical substance is used in combination, not with nanobubbles, but with bubbles of a comparatively large diameter is known being disclosed in JP 2008-238165A. The invention disclosed in this patent application relates to a dispersing method for keeping stable a dispersion having a substance dispersed in a liquid, which is characterized by incorporating bubbles in the dispersion. However, the main thrust of this method is that a dispersion having improved stability is produced by causing bubbles to be present in the process of its production and the resulting dispersion has no bubbles present in it. This should be clear from the fact that in the invention disclosed in JP 2008-238165A, the preferred diameter of the bubbles to be used ranges from 30 to 1000 microns but that bubbles of 1000 microns (1 mm) are unable to exist stably in the dispersion for an extended period of time. In addition, the particle size of the bubbles greatly differs from that of the ultrafine bubbles to be used in the present invention and the effect of such bubbles is by no means satisfactory, as demonstrated by the Examples in which more than 10% of the dispersed oil separated in 48 hours.

### Patent Literature

Patent Document 1: JP 2008-238165A

### SUMMARY OF THE INVENTION

The present inventor found that a composition comprising novel ultrafine bubbles in the nano-range and a drug allowed the drug to exhibit its effect more pronouncedly and that when the drug was dispersed, a stable dispersion could be obtained without using a surfactant; the present invention has been accomplished on the basis of these findings.

The present invention relates to a composition comprising novel ultrafine bubbles in the nano-range and a drug, and a dispersion comprising novel ultrafine bubbles in the nano-range and a hydrophobic drug dispersed as particles. The present invention further relates to a detergent composition prepared from a specified recipe and a washing method that uses the detergent composition. The present invention also provides processes for producing the composition and the dispersion.

The present invention provides a composition comprising ultrafine bubbles having a mode particle size of no more than 500 nm and a drug, as well as water.

In a first mode of the present invention, the drug is a water-soluble drug and dissolved in the water.

In a second mode of the present invention, the drug is a hydrophobic drug and dispersed in the water. To be more specific, the hydrophobic drug is dispersed as dispersoid particles in the water which serves as the dispersion medium.

In the second mode of the present invention, mode particle size of the dispersed drug particles preferably ranges from 0.05 μm to 15 μm. The mean particle size of the dispersed drug particles may also preferably range from 0.05 μm to 15 μm. Depending on the type of the hydrophobic drug to be dispersed, there can be formed such fine drug particles that their mode particle size and/or mean particle size ranges from 0.05 μm to 3 μm. Note that the “hydrophobic drug” as used herein refers to a drug that is poorly soluble in water but which is oil-soluble.

The aforementioned ultrafine bubbles have a mode particle size of no more than 500 nm, preferably no more than 300 nm, and most preferably no more than 150 nm, and they are present at a density of at least 1×10⁶, preferably at least 3×10⁶, more preferably at least 4×10⁶, and most preferably at least 5×10⁶ bubbles, per milliliter.

In one mode of the present invention, the surfaces of the ultrafine bubbles contained in the composition or dispersion are electrically charged to provide zeta potential that are at least 5 mV in absolute value.

In a preferred mode, the drug is an evaporative substance. In a more preferred mode, the evaporative substance is at least one substance selected from the group consisting of insecticides, bactericides, repellents, allergen inactivators, deodorants, antifungal agents, fragrances (air fresheners), essential oils, and flavorings.

The composition or dispersion of the present invention need not be a liquid and may instead be in a gel form. To render the dispersion into a gel form, agar, carrageenan, gelatin, water absorbing resins, aqueous polymers, etc. may be used. For example, carrageenan is added to distilled water and the mixture is heated to prepare a carrageenan solution, which is mixed well under agitation with the composition comprising the fine bubbles, drug and water. The resulting mixture is cooled to room temperature to thereby form a gelled dispersion. If desired, the dispersion may be converted to a mist by using an atomizer.

The present invention further provides a detergent composition that contains at least one gas in the ultrafine bubbles as selected from the group consisting of air, oxygen, hydrogen and nitrogen, with alkali electrolyzed water being used as the water and at least one compound selected from among terpenes being used as the drug; the present invention also provides a washing method that uses the detergent composition with ultrasonic waves being applied.

The present invention further provides a process for preparing a composition comprising ultrafine bubbles having a mode particle size of no more than 500 nm, water and a water-soluble drug dissolved in water, wherein ultrafine bubbles having a mode particle size of no more than 500 nm is generated in a solution of the water-soluble drug and water by means of an ultrafine bubble generator.

The present invention further provides a process for preparing a composition comprising ultrafine bubbles having a mode particle size of no more than 500 nm, water and a hydrophobic drug dispersed in water, wherein ultrafine bubbles having a mode particle size of no more than 500 nm is generated in a dispersion of water and the hydrophobic drug by means of an ultrafine bubble generator.

The present invention further provides a process for preparing a composition comprising ultrafine bubbles having a mode particle size of no more than 500 nm, water and a hydrophobic drug dispersed in water, wherein ultrafine bubbles having a mode particle size of no more than 500 nm is generated in water by means of an ultrafine bubble generator, after that the hydrophobic drug is added to water comprising the ultrafine bubbles.

Since it contains the ultrafine bubbles, the composition of the present invention allows the drug to exhibit its effect more pronouncedly. For example, if the drug is evaporative, its evaporation is improved and its concentration in the composition can be reduced accordingly. If the drug is an antifungal agent or the like, its penetrability is improved to provide a greater effect. Conventionally, evaporation has been accomplished by various methods including thermal evaporation, volatilization of the drug by wind, evaporation by an ultrasonic vibrator, etc.; however, the thermal method has the disadvantage of not permitting the use of substances that have a tendency to decompose with heat and, in addition, since the respective methods use a heating device, a fan, and an ultrasonic vibrator, they all suffer from an increases cost of manufacturing the evaporation apparatus and entail the operating cost. In contrast, the present invention is not only safe and low in the cost of manufacturing the apparatus but also requires no operating cost, with the additional advantage of being applicable to a wide range of substances in a safe manner.

If a hydrophobic drug is dispersed in water, the present invention offers the advantage of providing a dispersion that remains stable for an extended period of time without using a surfactant. Since no surfactant is used, cost reduction is possible and there is no need for treating waste liquid that would otherwise occur if a surfactant were used. Particularly in the case where the diameter of particles is decreased in order to improve the stability of the dispersion, it has been necessary to use a large amount of surfactant; in the present invention, however, there is no need to use a surfactant, so not only further cost reduction is achieved but also the problem of a decrease in the available content of the actually dispersed substance on account of the increased use of a surfactant can be solved.

### BRIEF DESCRIPTION OF THE DRAWINGS

FIG. 1 shows the size distribution of freshly generated ultrafine bubbles for use in the present invention and the change in it until after the lapse of 3 months (as measured with Multisizer 3).

FIG. 2 shows the result of measurement of the particle diameter of a sample of the ultrafine bubbles to be used in the present invention (as measured with the nanoparticle size analyzing system: NanoSight Series).

FIG. 3 shows the result of measurement of the particle diameter of another sample of the ultrafine bubbles to be used in the present invention (as measured with the nanoparticle size analyzing system: NanoSight Series).

FIG. 4 shows the result of measurement of the zeta potential on the ultrafine bubbles to be used in the present invention (as measured with ELSZ-1 of OTSUKA ELECTRONICS CO., LTD.)

FIG. 5 shows graphically the particle size distribution of the emulsion as freshly prepared in Example 2 (the measurement conducted with the particle size distribution analyzer LS 13 320).

FIG. 6 shows graphically the particle size distribution of the emulsion that was prepared in Example 2 and stored at room temperature for 3 months (the measurement conducted with the particle size distribution analyzer LS 13 320).

FIG. 7 shows graphically the particle size distribution of the emulsion that was prepared in Example 2 and stored at 40 C for 3 months (the measurement conducted with the particle size distribution analyzer LS 13 320).

FIG. 8 shows graphically the particle size distribution of the dispersion as freshly prepared in Example 3 (the measurement conducted with the particle size distribution analyzer LS 13 320).

FIG. 9 shows graphically the particle size distribution of the dispersion that was prepared in Example 3 and stored at room temperature for 2 months (the measurement conducted with the particle size distribution analyzer LS 13 320).

FIG. 10 shows graphically the particle size distribution of the dispersion that was prepared in Example 3 and stored at 40° C. for 2 months (the measurement conducted with the particle size distribution analyzer LS 13 320).

FIG. 11 shows graphically the particle size distribution of the dispersion as freshly prepared in Example 4 (the measurement conducted with the particle size distribution analyzer LS 13 320).

FIG. 12 shows graphically the particle size distribution of the dispersion that was prepared in Example 4 and stored at room temperature for 2 months (the measurement conducted with the particle size distribution analyzer LS 13 320).

FIG. 13 shows graphically the particle size distribution of the dispersion that was prepared in Example 4 and stored at 40° C. for 2 months (the measurement conducted with the particle size distribution analyzer LS 13 320).

### DETAILED DESCRIPTION OF THE PREFERRED EMBODIMENTS

The present invention provides a composition comprising ultrafine bubbles having a mode particle size of no more than 500 nm and a drug, as well as water.

The particle diameter of the ultrafine bubbles to be used in the present invention is so small that it cannot be measured correctly with an ordinary particle size distribution analyzer. Hence, hereinafter, numerical values are employed that were obtained by measurements with the nanoparticle size analyzing system NanoSight Series (product of NanoSight Ltd.). The nanoparticle size analyzing system NanoSight Series (product of NanoSight Ltd.) measures the velocity of nanoparticles moving under Brownian motion and calculates the diameters of the particles from the measured velocity. A mode particle size can be verified from the size distribution of the particles present. The interior of the ultrafine bubbles is generally filled with air, which may be replaced by other gases including oxygen, hydrogen, nitrogen, carbon dioxide, and ozone.

The drug may be any compound that works effectively for a desired object. From the aspect of chemical structure, the drug may be exemplified by but are not limited to water-soluble substances such as various water-soluble natural substances, lower alcohols, glycols, esters, acids, bases, salts, and water-soluble polymers and water-soluble proteins, as well as hydrophobic substances such as plant-derived oils, animal-derived oils, lipids, hydrocarbons, waxes, esters, fatty acids, higher alcohols, non-water-soluble polymers, oil-soluble pigments, and oil-soluble proteins. From the functional aspect, the drug may be exemplified by but are not limited to various pharmaceuticals, cosmetics, insecticides, bactericides, agrichemicals, fertilizers, vitamins, paints, adhesives, and wetting agents.

Water that can be used may be exemplified by distilled water, ultrapure water, highly pure water, pure water, tap water, ion-exchanged water deionized water, filtered water, electrolyzed water, and natural water. If performance is not compromised, a water-miscible solvent such as alcohol may be contained as a co-solvent in a small quantity.

In the first mode of the present invention, the above-described drug is dissolved in water. While any water-soluble drugs may be used, preferred water-soluble drugs to be used in this mode include, for example, antifungal agents, fragrances, allergen inactivators, deodorants, bactericides, and repellents. Exemplary water-soluble drugs include the following: sodium hypochlorite, chlorinated lime, mercurochrome solution, alcohols (e.g. ethanol and isopropanol), hydrogen peroxide, invert soaps (e.g. benzalkonium chloride and cetyl pyridinium chloride), surfactants, phenols (e.g. cresol soap solution), diphenols such as catechol, 4-methylcatechol, 5-methylcatechol, resorcinol, 2-methylresorcinol, 5-methylresorcinol, and hydroquinone, polyhydroxyamine compounds such as 4,4′-biphenyldiol and 3,4′-diphenyldiol, dopa, dopamine, caffeic acid, paracoumaric acid, tyrosine, ethanolamine, triethanolamine, and tris(hydroxymethyl)aminomethane, or polyphenols including flavones (apigenin, luteolin, tangeritin, diosmin, and flavoxate), isoflavones (coumesterol, daizein, daizin, and genistein), flavanols (kaempferol, myricetin, and quercetin), flavanones (eriodictyol, hesperetin, homoeriodictyol, and naringenin), flavan-3-ols (catechin, epicatechin, and epigallocatechin), anthocyanidins (e.g. cyanidin, as well as delphinidin, malvidin, pelargonidin, and peonidin), phenolic acid, chlorogenic acid, ellagic acid, lignan, curcumin, hydroquinone derivatives, kojic acid, L-ascorbic acid and derivatives thereof, tranexamic acid and derivatives thereof, glycyrrhizinates, resorcin, salicylic acid, chlorhexidine gluconate, vitamin B₆ and derivatives thereof, nicotinic acid and derivatives thereof, pantothenyl ethyl ether, trypsin, hyaluronidase, thiotaurine, glutathione, piperine, fruit juice, glucose, as well as water-soluble plant extracts including rosemary extract, lemon extract, Litchi chinensis extract, Momordica charantia var. pavel extract, glucosamine, star flute extract, Alpinia zerumbet extract, Ginkgo biloba extract, trehalose, kaki (Japanese persimmon) extract, lavender extract, wormwood extract, peach leaf extract, sage extract, pine extract, Luffa cylindrica (L.) Roem. extract, carrot extract, Angelica acutiloba root extract, tomato extract, red pepper extract, aloe extract, seaweed extract, sage extract, Eugena caryophyllus (clove) flower extract, corn maize extract, thyme extract, eucalyptus leaf extract, Cupressus sempervirens extract, savory extract, clove extract, mint extract, pepper extract, tea extract, Rosa roxburghii extract, and sugar cane liquid extract; some of these extracts may be used in combination. It should be mentioned that the foregoing examples are non-limiting and the scope of the present invention is by no means limited to those compounds.

In the second mode of the present invention, the aforementioned drug is dispersed in water.

In this mode, the drug forms a discontinuous phase as the dispersoid whereas water forms a continuous phase as the dispersion medium. Preferred hydrophobic drugs to be used in this mode may include, for example, insecticides, bactericides, repellents, allergen inactivators, deodorants, antifungal agents, fragrances (air fresheners), essential oils, and flavorings. Exemplary hydrophobic drugs include the following: pyrethroid agents (pyrethrin, permethrin, etofenprox, etc.), organophosphorus agents (parathion, dichlorvos, malathion, fenitrothion, etc.), carbamate agents (carbaryl, propoxer, fenobucarb, etc.), chloronicotinyl agents (imidachloprid, acetamiprid, dinotefuran, etc.), iodine agents (iodine tincture and povidone iodine), triclosan, isopropyl methylphenol, acrinol, diethylamide.di-N-propyl isocinchomeronate, 2.3.4.5-bis(Δ₂-butylene)tetrahydrofurfural, dinormalpropyl isocinchomeronate, N-octyl-bicycloheptene.dicarboximide, β-naphthol, as well as cycloheximide, acetyl-iso-eugenol, anethole, iso-amyl acetate, allylamyl glycolate, allyl heptanoate, aldehyde C-14 peach, aldehyde C-16 strawberry, estragole, eugenol, λ-carvone, camphor, camphene, iso-cyclocitral, 1,8-cineole, citral, citronellal, dimetol, dimethyl benzyl carbinyl acetate, α-damascone, β-damascone, δ-damascone, damascenone, terpineol, terpinyl acetate, terpinolene, terpinen-4-ol, thymol, o-t-butylcyclohexyl acetate, cis-3-hexenyl acetate, FRUITATE, POIRENATE, POLLENAL II, iso-bornyl acetate, p-methyl acetophenone, methyl-iso-eugenol, methyl ionone-γ, λ-e-menthol, menthone, iso-menthone, methyl salicylate, menthanyl acetate, lactone C-10 gamma, linalyl acetate, aldehyde C-11, aldehyde C-12 lauric, aldehyde C-12 MNA, ambroxan, amylcinnamic aldehyde, amyl salicylate, benzaldehyde, benzyl acetate, benzyl salicylate, cedrol, cinnamic alcohol, coumarin, cyclopentadecanolide, γ-decalactone, ethyl vanillin, eugenol, hexylcinnamic aldehyde, indole, α-ionone, isoeugenol, lilial, linalool, linalyl acetate, lyral, maltol, methyl anthranilate, methylionone, γ-methylionone, musk ketone, musk xylol, phenyl acetaldehyde, phenyl acetate, sulfur, phenylethyl alcohol, phenylpropyl alcohol, α-pinene, α-terpineol, tonalid, vanillin, and Vertofix Coeur, as well as essential oils including rosemary oil, lemon grass oil, mint oil, spearmint oil, sage oil, ginger oil, anise oil, armoise oil, estragon oil, cardamon oil, camphor oil, caraway oil, carrot seed oil, clove oil, coriander oil, citronella oil, spearmint oil, clary sage oil, thyme oil, pine oil, basil oil, fennel oil, volatile laurel oil, peppermint oil, lavandine oil, marjoram oil, lavender oil, laurel leaf oil, eucalyptus oil, and neem oil; oil-soluble plant extracts including tea extract, Rosa roxburghii extract, sugar cane extract, lemon extract, Litchi chinensis extract, Momordica charantia var. pavel extract, glucosamine, star fruit extract, Alpinia zerumbet extract, Ginkgo biloba extract, fruit juice, trehalose, kaki (Japanese persimmon) extract, lavender extract, wormwood extract, peach leaf extract, sage extract, pine extract, Luffa cylindrica (L.) Roem. extract, carrot extract, Angelica acutiloba root extract, tomato extract, red pepper extract, aloe extract, seaweed extract, sage extract, Eugena caryophyllus (clove) flower extract, corn maize extract, thyme extract, eucalyptus leaf extract, Cupressus sempervirens extract, savory extract, clove extract, mint extract, and pepper extract; as well as terpenes including terpene hydrocarbon such as pinene, menthene, cymene, phellandrene, menthane and limonene, and terepene alcohols such as citronellol, pinocampheol, gellaniol, fencyl alcohol, nerol, linalool, and borneol; some of these extracts may be used in combination. It should be mentioned that the foregoing examples are non-limiting and the scope of the present invention is by no means limited to those compounds.

If the hydrophobic drug is to be dispersed in water, the mode particle size of the drug particles preferably ranges from 0.05 μm to 15 μm, more preferably from 0.05 μm to 6μm. Depending on the type of the drug to be dispersed, there can be formed ultrafine drug particles in the range of 0.05 μm to 3 μm. The mean size of the drug particles may also preferably range from 0.05 μm to 15 μm, more preferably from 0.05 μm to 6 μm. Depending on the type of the hydrophobic drug to be dispersed, there can be formed ultrafine drug particles having a mean size in the range of 0.05 μm to 3 μm.

The size distribution of the dispersed drug particles as referred to in the present invention was measured with the particle size distribution analyzer LS 13 320 (product of BECKMAN COULTER). The mode particle size is a maximum value of particle diameter as expressed in percentages by volume or number and is also called the mode particle diameter. The mean size is a number average diameter or volume average diameter. Note that the size distribution data to be shown later in the Examples are assumed to represent the size distributions of drug particles surrounded with ultrafine bubbles on the surface, and the ultrafine bubbles themselves.

In the present invention, ultrafine bubbles occur at a density of at least 1×10⁶, preferably at least 3×10⁶, more preferably at least 4×10⁶, and most preferably at least 5×10⁶, per milliliter. The number of ultrafine bubbles as referred to in the present invention was measured with the nanoparticle size analyzing system NanoSight Series (product of NanoSight Ltd.)

In one mode of the present invention, there are provided a detergent composition that uses alkali electrolyzed water as the water and a terpene compound, preferably at least one compound selected from among terpene hydrocarbons and terpene alcohols, as the drug, and wherein at least one gas selected from the group consisting of air, hydrogen, oxygen and nitrogen is contained within ultrafine bubbles, as well as a washing method that uses this detergent composition with ultrasonic waves being applied.

Alkali electrolyzed water that can advantageously be used in the present invention has a pH of at least 10, preferably between 10 and 13. An Example of such alkali electrolyzed water is commercially available from FELICITY Co., Ltd. under the trade name “STRONG ALKALI WATER” with a pH of 11.7.

Examples of terpene hydrocarbons that can advantageously be used in the present invention include pinene, menthene, cymene, phellandrene, menthane, and limonene. Examples of terpene alcohols that can advantageously be used in the present invention include citronellol, pinocampheol, gellaniol, fencyl alcohol, nerol, and borneol; some of these extracts may be used in combination. It should be mentioned that the foregoing examples are non-limiting and the scope of the present invention is by no means limited to those compounds. It should also be mentioned that terpene hydrocarbons are preferably used and most preferably limonene is used.

The ultrafine bubbles may individually contain gaseous air, oxygen, hydrogen or nitrogen either independently or as a mixture of two or more gases. In the latter case, if hydrogen and nitrogen are used, bubbles that contain hydrogen may be present together with bubbles that contain nitrogen or, alternatively, bubbles that contain a gaseous mixture of hydrogen and nitrogen may occur. The most preferred effect is obtained when hydrogen is used as a gas. The mixing ratio of gases can be empirically determined as appropriate not only for achieving a maximum washing effect but also from the viewpoints of safety and cost.

The detergent composition of the present invention is advantageously used for removing rust and stain on metals, as well as stain that has deposited on plastic, cloth and various other substrates. Washing is advantageously performed with ultrasonic waves being generated in the detergent. For generating ultrasonic waves, a known device can be used and appropriate values of its operating frequency and intensity can be easily determined on an empirical basis. To “generate ultrasonic waves in the detergent,” the detergent may generally be fed into a bath equipped with a sonicator and any method may be adopted if ultrasonic wave irradiation to the detergent and/or the object being washed is possible.

Although not wishing to be bound by theory, the present inventor assumes that the superior effects of the present invention are achieved by the following mechanism. If the drug is water-soluble, the moving ultrafine bubbles would enhance the motion of the drug molecules to make them more efficacious and the ultrafine bubbles would themselves increase the penetration of the aqueous solution to exhibit even better effect. If the drug is hydrophobic and dispersed in water, the ultrafine bubbles would gather on the surfaces of the dispersed drug particles and the zeta potential on the bubble surfaces would create a sufficient surface active effect to stabilize the dispersed particles. Therefore, it is important that the number of ultrafine particles be kept within a preferred range.

From the viewpoints just described above, the zeta potential on the surfaces of the ultrafine particles contained in the composition or dispersion is also considered to be important for ensuring the present invention to exhibit its intended effects. The surfaces of the ultrafine particles used in the present invention are electrically charged to produce a zeta potential of at least 5 mV, preferably at least 7 mV, in absolute value. Since the absolute value of zeta potential is proportional to the viscosity/dielectric constant of the solution, the lower the temperature at which the ultrafine bubbles, drug and water are treated, the more likely it is that the resulting dispersion has higher stability.

The ultrafine particles to be used in the present invention that have a mode particle size of no more than 500 nm can be generated by any known means, such as the use of a static mixer, the use of a venturi tube, cavitation, vapor condensation, sonication, swirl formation, dissolution under pressure, or fine pore formation. A preferred method of bubble generation is by forming a gas-liquid mixture and shearing it.

An advantageous apparatus for generating ultrafine bubbles by the gas-liquid mix shearing method is disclosed in Japanese Patent No. 4118939. In this apparatus, the greater part of a gas-liquid mixture in fluid form introduced into a fluid swirling compartment does not simply flow toward the discharge port as in the apparatus described in the prior art section but it first flows forming a swirl in the direction away from the discharge port. The swirl reaching the first end-wall member turns around and flows back toward the second end-wall member; since the returning swirl has a smaller radius of rotation than the swirl flowing toward the first end-wall member, it flows at a higher velocity, creating a sufficient shear force on the gas within the liquid to promote the formation of finer bubbles.

If the drug is water-soluble, its aqueous solution is treated with a suitable apparatus to generate ultrafine bubbles in it, whereby the composition of the present invention can be produced that has the drug dissolved in the water.

If the drug is hydrophobic, a mixture of the hydrophobic drug and water is treated with a suitable apparatus to generate ultrafine bubbles in the aqueous dispersion of the hydrophobic drug, whereby the composition of the present invention can be produced that has the hydrophobic drug dispersed in the water. Alternatively, water may be treated with a suitable apparatus to generate ultrafine bubbles in it and thereafter the hydrophobic drug is added, whereby the composition of the present invention can be produced that has the hydrophobic drug dispersed in the water. Note that a hydrophobic drug that is solid at ordinary temperature may also be used if it is thermally melted or dissolved in a solvent.

There is no need to use a surfactant in the present invention but it should be appreciated by skilled artisans that this does not mean excluding the case of adding a surfactant as appropriate for use and other conditions.

The foregoing description of the present invention and the description of the Examples that follow are only intended to provide a detailed explanation of various exemplary embodiments of the present invention and skilled artisans can made various improvements and changes of the embodiments disclosed herein without departing from the scope of the present invention. Therefore, the description herein will in no way limit the scope of the present invention, which shall be determined only by the recitation in the appended claims.

### EXAMPLES

### Example 1

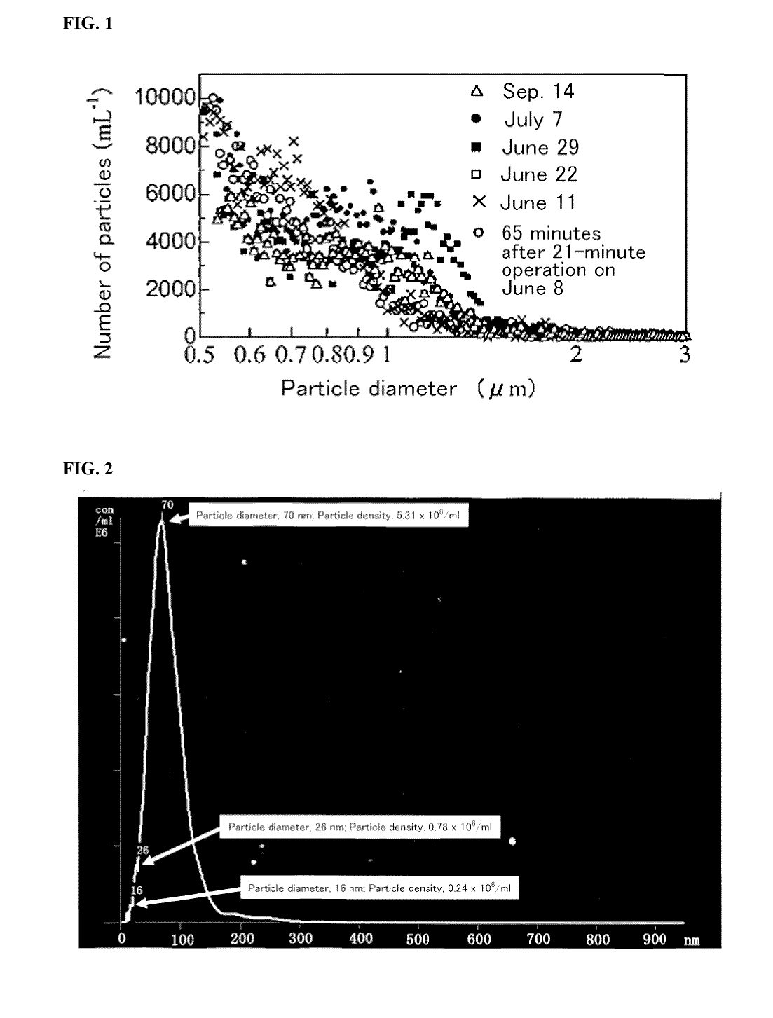

Ultrafine bubbles were generated in pure water having a resistivity of 18.2 MΩ/cm using BUVITAS of KYOWA KISETSU which was a device for generating ultrafine bubbles by the gas-liquid mix shearing method. FIG. 1 shows the size distribution of the freshly generated ultrafine bubbles and the change in it until after the lapse of 3 months. Size distribution was measured with Multisizer 3 (product of BECKMAN COULTER). Obviously, there was no change in the number of bubbles with diameters of no more than 1 μm.

At the same time, the diameters of the generated ultrafine bubbles were measured with the nanoparticle size analyzing system NanoSight Series (product of NanoSight). The results are shown in FIGS. 2 and 3. The horizontal axis of each graph represents the particle diameter in nanometers and the vertical axis represents the number of particles per millimeter (1×10⁶/ml ). FIG. 2 shows the result of a measurement that was conducted 24 hours after the generation of the ultrafine bubbles and FIG. 3 shows the result after the passage of 48 hours. It was verified that the bubbles had a mode particle size of no more than 500 nm, with 4 to 8×10⁶ counts per ml, and that the generated ultrafine bubbles remained stable in the water for an extended period.

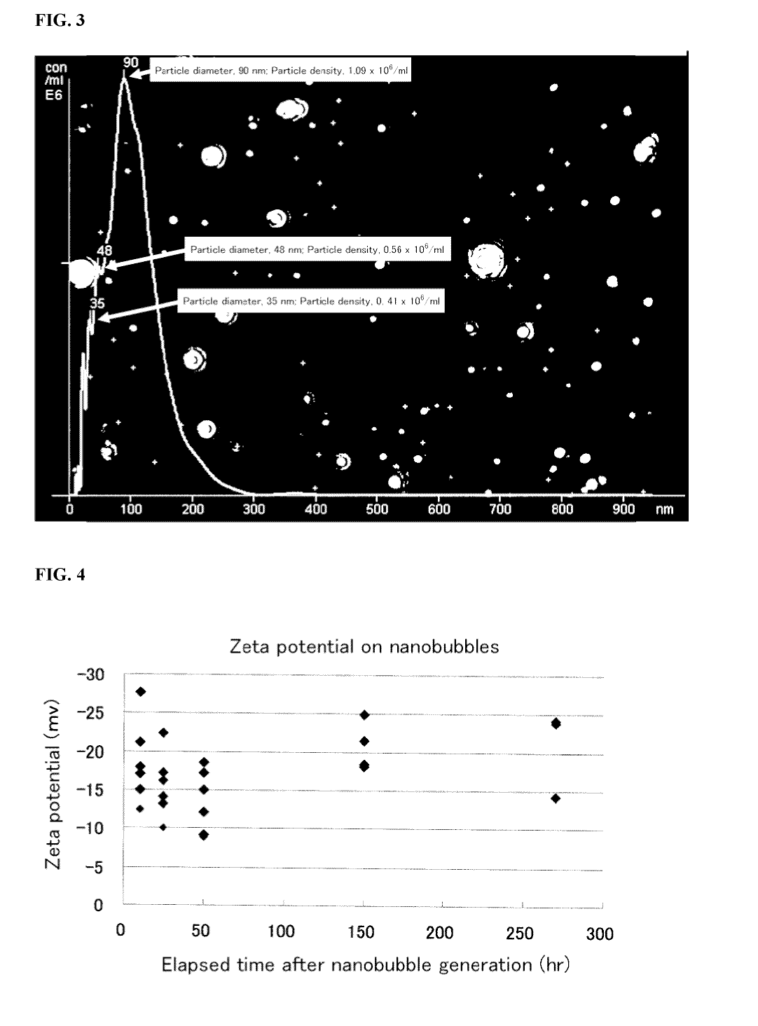

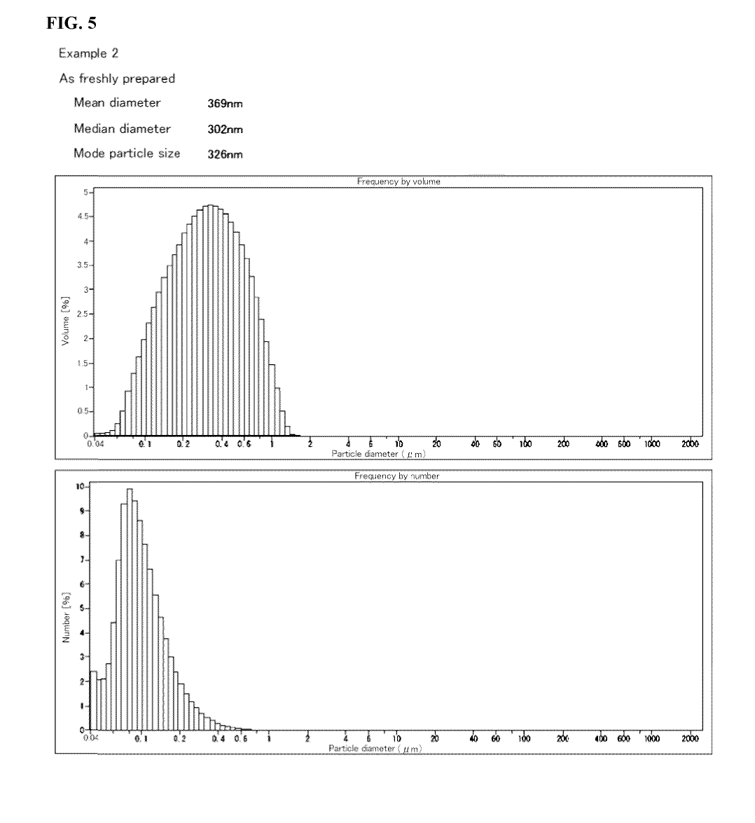

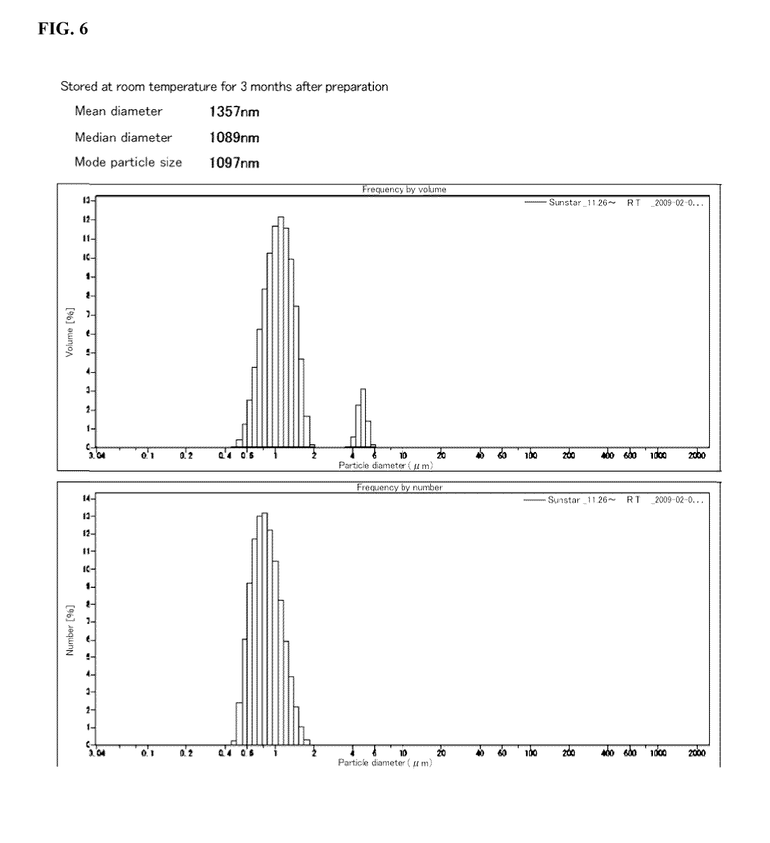

In addition, the zeta potential on the generated bubbles was measured with the zeta potential measuring system ELSZ-1 of OTSUKA ELECTRONICS CO., LTD. The result is shown in FIG. 4. Obviously, zeta potential was maintained for an extended period, indicating the stability of the bubbles.

### Examples 2-5

Using BUVITAS of KYOWA KISETSU, mixtures having the compositions shown in Table 1 below were treated under the same conditions that were used in Example 1, except that the pure water was replaced by distilled water. The results are shown in Table 1.

TABLE 1

| | | | | | | | Comp. |
|---|---|---|---|---|---|---|---|
| | | | Example 2 | Example 3 | Example 4 | Example 5 | Ex. 1 |
| Fine dispersion maker | | | Fine bubble generator having | | | | Homo- |
| | | | a gas-liquid mixture shearing device*¹ | | | | mixer |
| Liquid | Distilled water | | 100 | 100 | 100 | 100 | 100 |
| dispersion | Oil | Orange oil | 0.25 | | | | 0.25 |
| | component | Neem | | 0.25 | | | |
| | | extract | | | | | |
| | | Limonene | | | 0.3 | | |
| Particle | | just after | 0.08 | 0.8 | 1.2 | 0.07 | 0.4 |
| diameter | | preparation | | | | | |
| μm | RT | 30 days | 0.80 | 0.9 | 1.2 | 0.07 | — |
| State of | | just after | white | white | white | transparent | separated |
| emulsion | | preparation | translucent | translucent | translucent | | |
| | RT | 2 weeks | white | white | white | ″ | ″ |
| | | | translucent | translucent | translucent | | |
| | | 30 days | white | white | white | ″ | ″ |
| | | | translucent | translucent | translucent | | |
| | | 60 days | white | white | white | ″ | ″ |
| | | | translucent | translucent | translucent | | |
| | | 90 days | white | white | white | ″ | ″ |
| | | | translucent | translucent | translucent | | |
| | 40° C. | 30 days | white | white | white | ″ | ″ |
| | | | translucent | translucent | translucent | | |
| | | 60 days | white | white | white | ″ | ″ |
| | | | translucent | translucent | translucent | | |
| | | 90 days | white | white | white | ″ | ″ |
| | | | translucent | translucent | translucent | | |

*¹Fine bubble generator: BUVITAS of KYOWA KISETSU

Examples 2-4 showed that the hydrophobic drugs were stably dispersed. All samples, whether they were stored at room temperature (RT) or 40° C., retained a satisfactory state of emulsification.

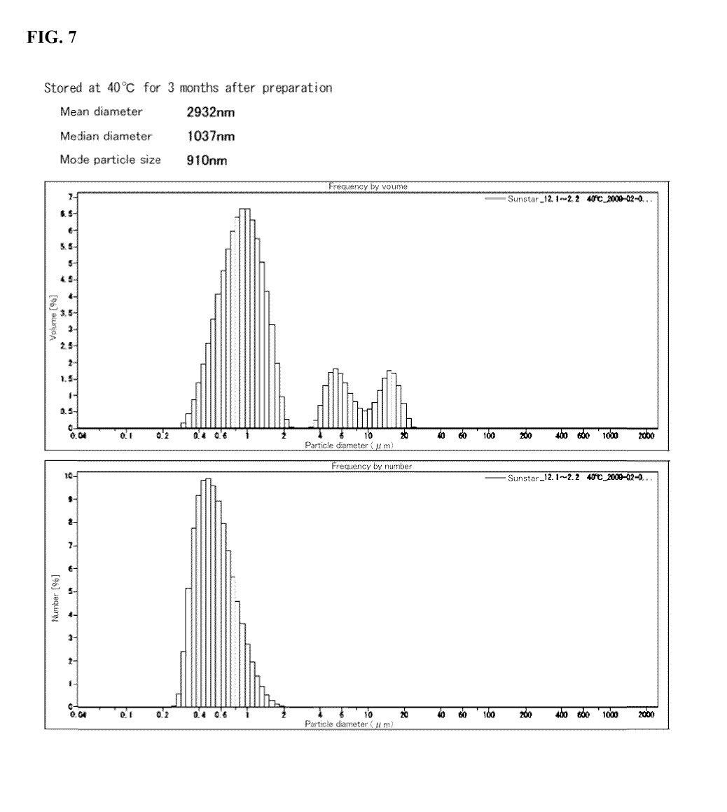

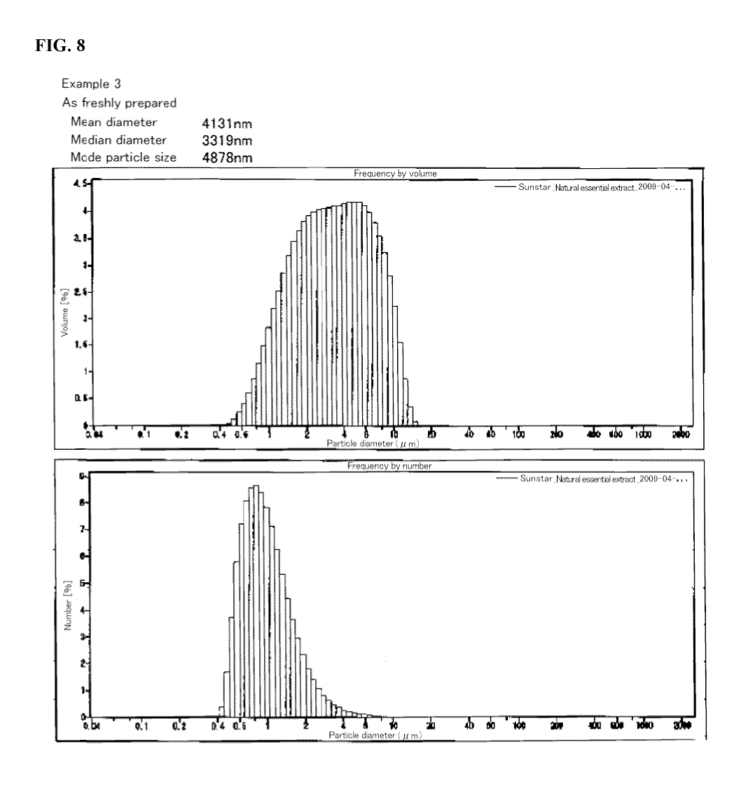

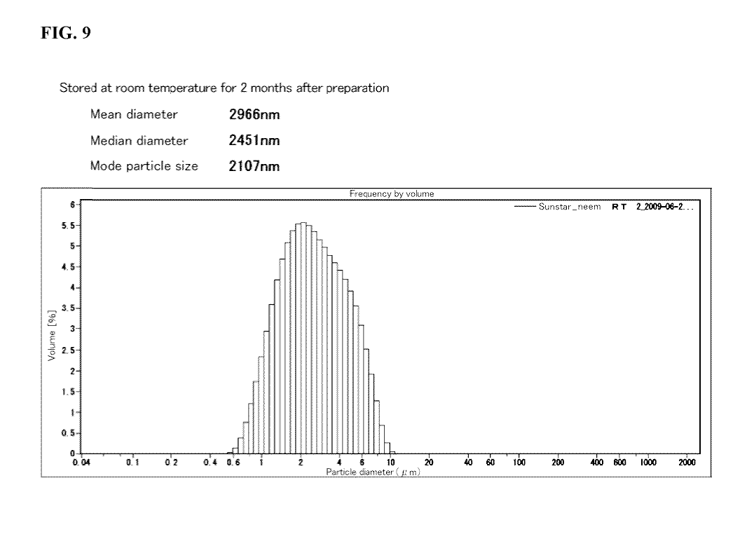

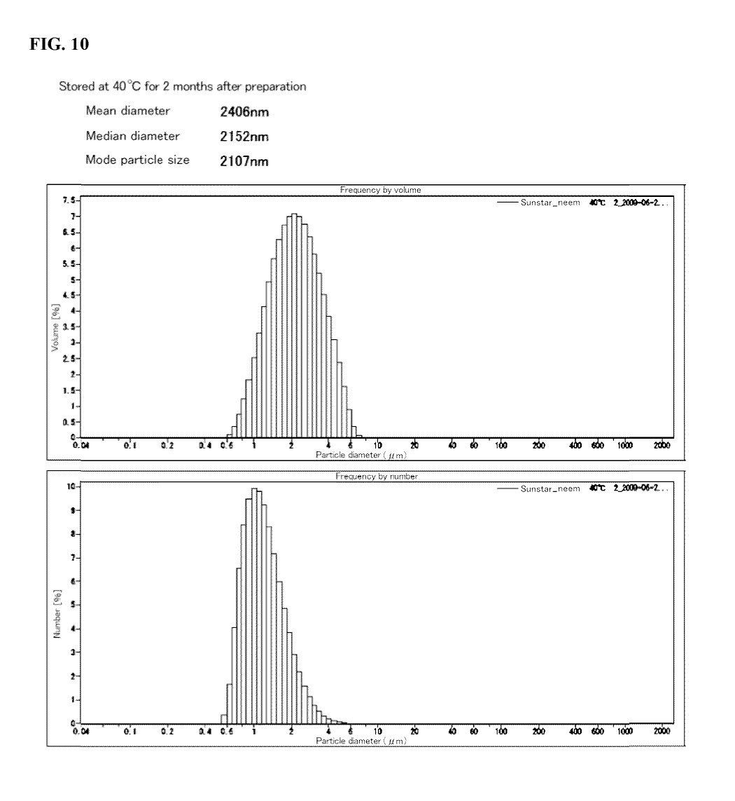

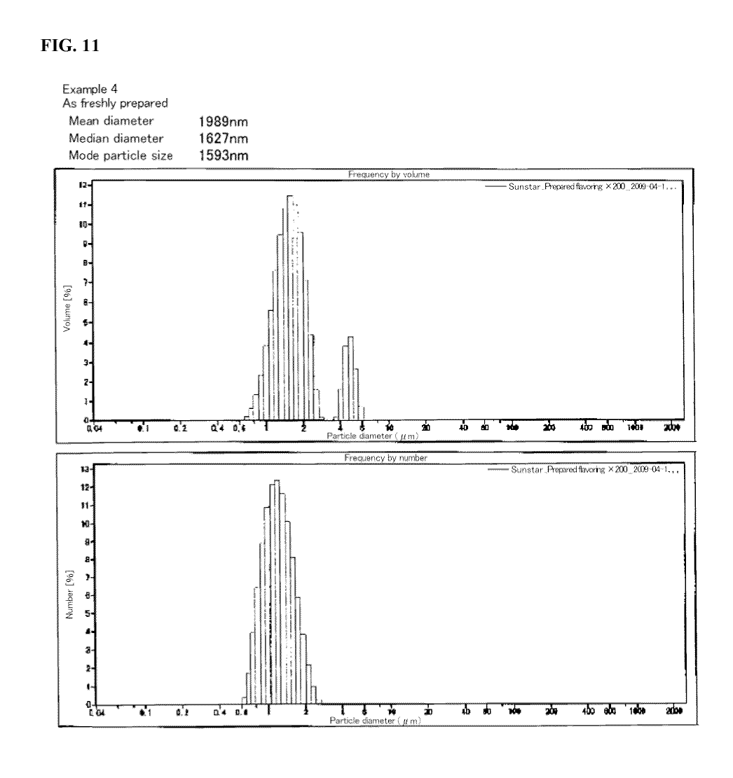

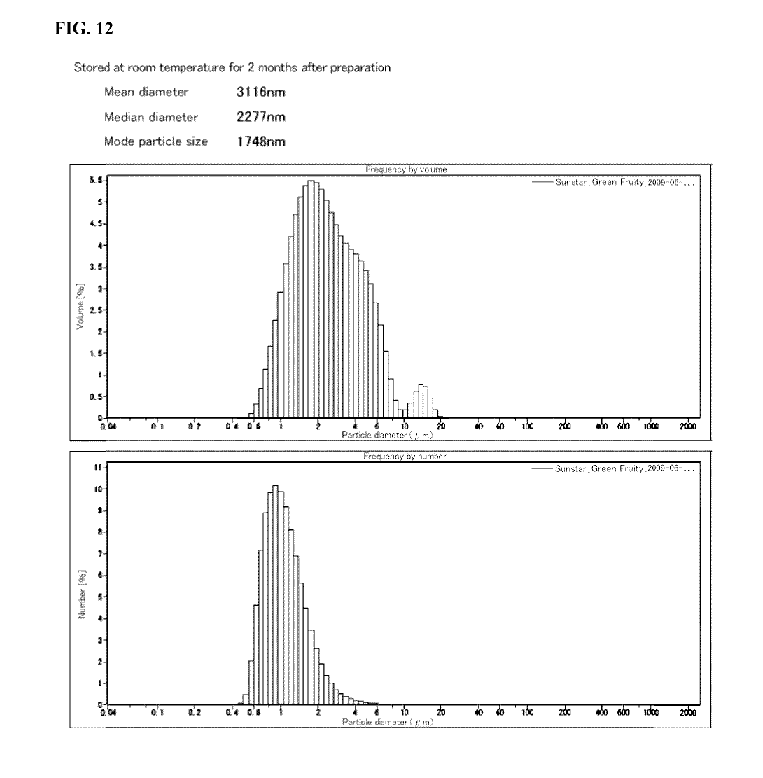

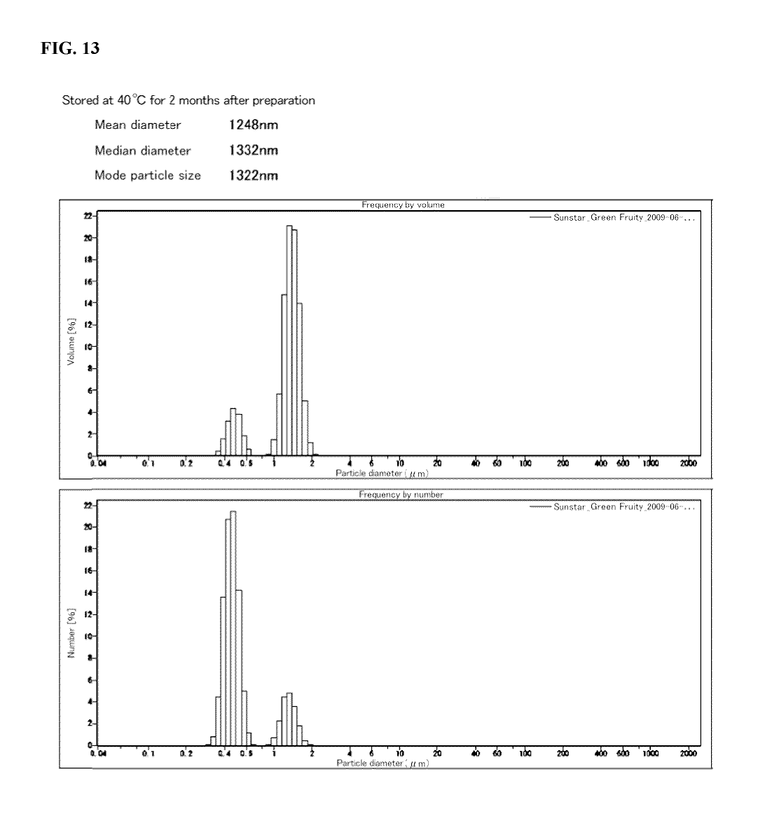

The particle size distributions of the dispersions prepared in Examples 2-4 were measured with the size distribution analyzer LS 13 320 (product of BECKMAN COULTER), both as freshly prepared and after storage at room temperature or 40° C.; the results are shown in FIGS. 5-13. The horizontal axis of each graph represents particle diameter and measurement was conducted for the volume and number percentages of each particle diameter, with the former being plotted in the upper panel and the latter in the lower panel. The values of mean size, median size and mode particle size in FIGS. 5-13 were calculated from volume percentages, and the data obtained from the sample of Example 3 after 2 month storage at room temperature were processed to measure only volume percentages. Although the particle diameter increased somewhat, the stability of dispersion was generally satisfactory in each of Examples 2-4.

As shown in Example 5, bubbles with a size of 70 nm were formed under the same experimental conditions. This would suggest that bubbles of approximately 70 nm in size were also formed in Examples 2-4.

As shown in Comparative Example 1, the dispersion prepared with a homomixer soon separated into two phases.

### Examples 6-8

To prepare samples for Examples 6-8, the components listed in Table 2 below were consecutively added in the amounts expressed in parts by weight in the same Table and the mixtures were treated with BUVITAS of KYOWA KISETSU under the same conditions as in Example 1, except that the pure water was replaced by distilled water.

In Comparative Examples 2 and 3, a surfactant was used to emulsify the same evaporative components as in Examples 6 and 7. In Comparative Example 4, the same drug as in Example 8 was dissolved using a homomixer.

TABLE 2

| | | | | | |
|---|---|---|---|---|---|
| | | | Comp. | Comp. | Comp. |
| Example 6 | Example 7 | Example 8 | Ex. 2 | Ex. 3 | Ex. 4 |

| | | |
|---|---|---|
| Fine dispersion maker | Fine bubble generator having | Homomixer |
| | a gas-liquid mixture shearing device*¹ | |

| Liquid | Distilled water | | 100 | 100 | 100 | 100 | 100 | 100 |
|---|---|---|---|---|---|---|---|---|
| dispersion | Evaporative | Limonene | 0.3 | | | 0.3 | | |
| | component | Antifungal | | 0.3 | | | 0.3 | |
| | | agent*² | | | | | | |
| | | Deodorant*³ | | | 0.25 | | | 0.25 |
| | Surfactant | Tween | | | | 0.1 | 0.1 | |

| | | | | | | | |
|---|---|---|---|---|---|---|---|
| Treatment conditions | Time | 10 min | 10 min | 10 min | 2 min | 2 min | 2 min |
| | Rotational | — | — | — | 20000 | 20000 | 20000 |
| | speed (rpm) | | | | | | |
| | Temperature | 10-20° C. | 10-20° C. | 10-20° C. | 10-20° C. | 10-20° C. | 10-20° C. |

*¹Fine bubble generator: BUVITAS of KYOWA KISETSU
*²Thiabendazole
*³Water-soluble liquid plant extract

### Example 6

Evaluation of Masking Performance:

Test method: In accordance with a modified odor bag method for odor sensory measurement, a test fluid was serially diluted with distilled water and subjected to sensory evaluation by 8 panelists for determining the thresholds of two liquid dispersions (threshold is a minimum limit of concentration that can be sensed by the human olfactory sense.)

How to Determine Thresholds:

The thresholds for the respective panelists were determined as common logarithms.

Xa=(log a₁+log a₂)/2 1)

wherein Xa: threshold for panelist A;

a₁: maximum dilution ratio at which panelist A gave the correct answer;

a₂: dilution ratio at which panelist A gave a wrong answer.

A maximum and a minimum value were excluded from the thresholds for the panelists and the intermediate other values were averaged to provide the threshold for the panel, X.

The value obtained from equation 1) was converted to the odor concentration by the following equation:

Y=10^{X} 2)

wherein X: threshold for the entire panel;

Y: odor concentration.

The results are shown in Table 3; ◯ means that the answer was correct and x means that the answer was wrong. The sample of Example 6 had a threshold approximately 10 times higher than the value for Comparative Example 2, indicating an improvement in the efficiency of flavoring's evaporation.

TABLE 3

| | Sample | Dilution ratio | | | | | | Threshold | Calculated |
|---|---|---|---|---|---|---|---|---|---|
| | to be | 1 * 10⁴ | 1 * 10⁵ | 3 * 10⁵ | 1 * 10⁶ | 3 * 10⁶ | 1 * 10⁷ | for the | threshold |
| Panelist | evaluated | 4.00 | 5.00 | 5.48 | 6.00 | 6.48 | 7.00 | panel | value |
| A | Comp. | ◯ | ◯ | ◯ | X | — | — | 5.74 | Comp. Ex. |
| | Ex. 2 | | | | | | | | 5.74 |
| | Example 6 | ◯ | ◯ | ◯ | ◯ | ◯ | X | 6.74 | Example |
| B | Comp. | ◯ | X | — | — | — | — | 4.50 | 6.99 |
| | Ex. 2 | | | | | | | | |
| | Example 6 | ◯ | ◯ | ◯ | ◯ | ◯ | ◯ | ≧7.24 | |
| C | Comp. | ◯ | ◯ | ◯ | ◯ | ◯ | | 6.74 | |
| | Ex. 2 | | | | | | | | |
| | Example 6 | ◯ | ◯ | ◯ | ◯ | ◯ | ◯ | ≧7.24 | |
| D | Comp. | ◯ | ◯ | ◯ | ◯ | X | — | 6.24 | |
| | Ex. 2 | | | | | | | | |
| | Example 6 | ◯ | ◯ | ◯ | ◯ | ◯ | ◯ | ≧7.24 | |
| E | Comp. | ◯ | ◯ | X | — | — | — | 5.24 | |
| | Ex. 2 | | | | | | | | |
| | Example 6 | ◯ | ◯ | ◯ | ◯ | ◯ | ◯ | ≧7.24 | |
| F | Comp. | ◯ | ◯ | ◯ | X | — | — | 5.74 | |
| | Ex. 2 | | | | | | | | |
| | Example 6 | ◯ | ◯ | ◯ | ◯ | X | — | 6.24 | |
| G | Comp. | ◯ | ◯ | ◯ | X | — | — | 5.74 | |
| | Ex. 2 | | | | | | | | |
| | Example 6 | ◯ | ◯ | | — | — | — | 5.24 | |
| H | Comp. | ◯ | ◯ | ◯ | X | — | — | 5.74 | |
| | Ex. 2 | | | | | | | | |
| | Example 6 | ◯ | ◯ | ◯ | ◯ | ◯ | ◯ | ≧7.24 | |

### Example 7

Evaluation of Antifungal Performance:

Test method: JIS Z 2911 Fungi Resistance Test Method, 8. Paint Test, with some modification. Two test fungi were used, Penicillium funiculosum and Alternaria alternata. The results are shown in Table 4.

TABLE 4

| Sample | Day 1 | | Day 2 | | Day 4 | |
|---|---|---|---|---|---|---|
| to be | Penicillin | Alternaria | Penicilliun | Alternaria | Penicilliun | Alternaria |
| evaluated | funiculosum | alternate | funiculosum | alternate | funiculosum | alternata |
| Comp. Ex. 3 | 0 | 0 | 0 | 4 | 4 | — |
| Example 7 | 0 | 0 | 0 | 2 | 2 | — |

Criteria for Evaluation:

0: No hyphae were observed.

1: Partial hyphal growth (less than ⅔ area coverage) was observed but no spores were found.

2: Partial hyphal growth (less than ⅔ area coverage) was observed, together with spores.

3: Hyphae were observed throughout (⅔ or more area coverage).

4: Spores were observed throughout (⅔ or more area coverage).

The sample of Example 7 showed higher fungal growth inhibitory performance than that of Comparative Example 3.

### Example 8

Evaluation of Odor Inhibiting Performance:

Test method: Filter paper impregnated with a malodor component (tobacco smell) was placed in a closed container and the malodorous substance was fully vaporized. A test liquid was sprayed in a metered amount into the container by means of a trigger spray and, one minute later, the intensity of the malodor component was evaluated by four sensory panelists on a three-point scoring scale, with 1 representing “the least intense,” 2, “moderate” and 3, “the most intense”.

TABLE 5

| | Blank | Comp. Ex. 4 | Example 8 |
|---|---|---|---|
| n = 1 | 3 | 2 | 1 |
| n = 2 | 3 | 2 | 1 |
| n = 3 | 3 | 2 | 1 |
| n = 4 | 3 | 2 | 1 |
| Average | 3 | 2 | 1 |

Obviously, the sample of Example 8 was less malodorous than that of Comparative Example 4.

### Examples 9 and 10

Mixtures having the compositions listed in Table 6 below were treated with BUVITAS of KYOWA KISETSU under the same conditions as in Example 1. In Example 10, deionized water was used after treatment with BUVITAS to generate ultrafine bubbles. Into the thus prepared ultrafine bubble-containing deionized water, λ-menthol was by means of a homomixer. In Comparative Examples 5 and 6, deionized water was also used but it was free from ultrafine bubbles; l-menthol was emulsified in this deionized water by means of a homomixer.

The results are shown in Table 6.

TABLE 6

| | | | | | Comp. | Comp. |
|---|---|---|---|---|---|---|
| | | | Example 9 | Example 10 | Ex. 5 | Ex. 6 |
| Ultrafine dispersion maker | | | | Homomixer*² | Homomixer | Homomixer |
| | Deionized water | | | | | |
| Liquid | Hydrophobic | l-menthol | 0.1 | 0.1 | 0.1 | 0.1 |
| dispersion | component | | | | | |
| | Surfactant | polyoxyethylene | | | | 0.1 |
| | | castor oil | | | | |

| | | | | | |
|---|---|---|---|---|---|
| Treatment | Time | 15 min | 15 min | 15 min | 15 min |
| conditions | Rotational speed (rpm) | — | 8000 | 8000 | 8000 |
| | Temperature | RT | RT | RT | RT |
| Results of | State of emulsion | white | white | white | white |
| evaluation | (just after preparation) | translucent | translucent | translucent | translucent |
| | State of emulsion | white | white | separated | white |
| | (day after preparation) | translucent | translucent | | translucent |

*¹Fine bubble generator: BUVITAS of KYOWA KISETSU
*²Deionized water containing ultrafine bubbles was used.

The sample of Example 9 maintained a more satisfactory state of emulsification than the sample of Comparative Example 5 which was simply treated with the homomixer. Good deionized water was also achieved in the sample of Example 10 which was prepared by adding l-menthol to the deionized water that was previously treated with BUVITAS of KYOWA KISETSU to generate ultrafine bubbles.

The compositions prepared in Examples 9 and 10 as well as Comparative Example 5 were evaluated for their masking performance.

Test method: In accordance with a modified odor bag method for odor sensory measurement, the samples identified in Table 6 (uniform dispersion just after preparation) were serially diluted with distilled water and subjected to sensory evaluation by 4 panelists for determining the thresholds of the samples. The procedure of threshold determination was the same as in Example 6. The results are shown in Table 7.

TABLE 7

| | | Dilution ratio | | | | | | | |
|---|---|---|---|---|---|---|---|---|---|
| | | 1 * 10⁴ | 3 * 10⁴ | 1 * 10⁵ | 3 * 10⁵ | 1 * 10⁶ | Threshold | | Threshold |
| Sample | Panelist | 4.00 | 4.48 | 5.00 | 5.48 | 6.00 | | Average | ratio |
| Example 9 | n = 1 | ◯ | ◯ | ◯ | X | — | 5.24 | 5.365 | 1 * 10^{5.37} |
| | n = 2 | ◯ | ◯ | ◯ | X | — | 5.24 | | |
| | n = 3 | ◯ | ◯ | ◯ | X | — | 5.24 | | |
| | n = 4 | ◯ | ◯ | ◯ | ◯ | X | 5.74 | | |
| Example 10 | n = 1 | ◯ | ◯ | X | — | — | 4.74 | 4.615 | 1 * 10^{4.62} |
| | n = 2 | ◯ | X | — | — | — | 4.24 | | |
| | n = 3 | ◯ | ◯ | X | — | — | 4.74 | | |
| | n = 4 | ◯ | ◯ | X | — | — | 4.74 | | |
| Comp. Ex. 5 | n = 1 | X | — | — | — | — | 3.74 | 4.115 | 1 * 10^{4.12} |
| | n = 2 | ◯ | X | — | — | — | 4.24 | | |
| | n = 3 | ◯ | X | — | — | — | 4.24 | | |
| | n = 4 | ◯ | X | — | — | — | 4.24 | | |

In Table 7, ◯ means that the answer was correct and x means that the answer was wrong. The samples of Examples 9 and 10 had higher thresholds than the sample of Comparative Example 5, indicating an improvement in the efficiency of l-menthol's evaporation.

Evaluation of Antifungal Performance:

Test method: A suspension of spores was prepared and smeared on Petri dishes each containing a potato dextrose agar medium. Sheets of filter paper (2.5 cm×2.5 cm) impregnated with the samples identified in Table 6 (uniform dispersion just after preparation) were attached to the inner surfaces at the center of a lid of the Petri dishes and culture was performed for 5 days at 23° C. and 100% RH. The test organism was Cladosporium cladosporioides, which was conditioned to form approximately 1×10² spores per ml.

After the culture, the state of fungal growth was observed visually and evaluated in accordance with the criteria adopted in Example 7 for evaluation of antifungal performance. The results are shown in Table 8.

TABLE 8

| Sample | Result |
|---|---|
| Example 9 | 1 |
| Example 10 | 1 |
| Comp. Ex. 5 | 2 |
| Comp. Ex. 6 | 2 |

Obviously, the compositions of Examples 9 and 10 were capable of more effective fungal inhibition than those of Comparative Examples 5 and 6.

### Example 11

A washing test was conducted using samples of washing water having the formulations identified in Table 9. A commercial product of artificially contaminated cloth was irradiated with supersonic waves for 3 hours as it was immersed in each of the washing water samples, and the state of the cloth's contamination was visually evaluated both before and after the washing.

Ultrasonic waves were generated with USD-4R, a sonicator manufactured by AS ONE Corporation, and its operating frequency was 28 kHz. The alkali electrolyzed water was “STRONG ALKALI WATER” available from FELICITY Co., Ltd. and having pH of 11.7. Limonene was used as a terpene and one of the gases filled in the ultrafine bubbles was a 1:24 mixture of H₂/N₂.

The results are shown in Table 10.

TABLE 9

| | Sample | | | |
|---|---|---|---|---|
| | No. | Base | Gas in bubbles | Functional agent |
| Example | [symbol] | Alkali electrolyzed water | Atmospheric air | Limonene |
| | | | | (0.3 wt % of the composition |
| | [symbol] | Alkali electrolyzed water | N₂ | Limonene |
| | | | | (0.3 wt % of the composition |
| | [symbol] | Alkali electrolyzed water | O₂ | Limonene |
| | | | | (0.3 wt % of the composition |
| | [symbol] | Alkali electrolyzed water | H₂ | Limonene |
| | | | | (0.3 wt % of the composition |
| | [symbol] | Alkali electrolyzed water | H2/N₂ = (1/24) | Limonene |
| | | | | (0.3 wt % of the composition |
| Com. | [symbol] | Alkali electrolyzed water | CO₂ | Limon e ne |
| Ex. | | | | (0.3 wt % of the composition |
| | [symbol] | Distilled water | N₂ | Limonene |
| | | | | (0.3 wt % of the composition |
| | [symbol] | Alkali electrolyzed water | None | Limonene |
| | | | | (0.3 wt % of the composition |

TABLE 10

| | Sample | | Washing |
|---|---|---|---|
| | No. | Dispersibility | performance |
| Example | [symbol] | ◯ | ◯ |
| | [symbol] | ◯ | ◯ |
| | [symbol] | ◯ | ◯ |
| | [symbol] | ◯ | ⊚ |
| | [symbol] | ◯ | ⊚ |
| Com. | [symbol] | X | X |
| Ex. | [symbol] | ◯ | X |
| | [symbol] | S | X |

In Table 10, the double circle represents “very good”, the single circle “good”, and x “poor.”

It was clear from the experimental results that great washing effects were obtained by causing nanobubbles and a terpene drug to be present in alkali electrolyzed water.

It was also shown that even when the terpene drug was present, the result was poor when distilled water was used or nanobubbles were absent, thus demonstrating that satisfactory washing performance was obtained only when the three conditions were met.

What is claimed is:

1. A composition comprising ultrafine bubbles having a mode particle size of no more than 500 nm and a drug, as well as water.

2. The composition according to claim 1, wherein the drug is dissolved in the water.

3. The composition according to claim 1, wherein the drug is dispersed in the water.

4. The composition according to claim 3, wherein the dispersed drug particles have a mode particle size in the range from 0.05 μm to 15 μm.

5. The composition according to claim 3, wherein the dispersed drug particles have a mean particle size in the range from 0.05 μm to 15 μm.

6. The composition according to claim 1, wherein the ultrafine bubbles are present at a density of at least 1×10⁶ per milliliter.

7. The composition according to claim 1, wherein the surfaces of the ultrafine bubbles contained in the composition or dispersion are electrically charged to provide zeta potentials that are at least 5 mV in absolute value.

8. The composition according to claim 1, wherein the drug is an evaporative substance.

9. The composition according to claim 8, wherein the evaporative substance is at least one substance selected from the group consisting of insecticides, bactericides, repellents, allergen inactivators, deodorants, antifungal agents, fragrances, essential oils, and flavorings.

10. The composition according to claim 1, wherein the composition is in a gel form.

11. The composition according to claim 1, which contains at least one gas in the ultrafine bubbles as selected from the group consisting of oxygen, hydrogen, nitrogen, carbon dioxide, and ozone.

12. A detergent composition comprising ultrafine bubbles having a mode particle size of no more than 500 nm, at least one compound selected from among terpenes, and alkali electrolyzed water, the ultrafine bubbles containing at least one gas selected from the group consisting of air, hydrogen, oxygen, and nitrogen.

13. A washing method that comprises holding an article of interest within the composition according to claim 12 and applying ultrasonic waves to the composition.

14. A process for preparing a composition according to claim 2, the process comprises to generate ultrafine bubbles having a mode particle size of no more than 500 nm in a solution of a water-soluble drug and water by means of an ultrafine bubble generator.

15. A process for preparing a composition according to claim 3, the process comprises to generate ultrafine bubbles having a mode particle size of no more than 500 nm in a dispersion of water and a hydrophobic drug by means of an ultrafine bubble generator.

16. A process for preparing a composition according to claim 3, the process comprises to add a hydrophobic drug to water comprising the ultrafine bubbles after generating ultrafine bubbles having a mode particle size of no more than 500 nm in water by means of an ultrafine bubble generator.

17. The process according to claim 14, wherein the ultrafine bubble generator is a gas-liquid mixture shearing device.
